(19) United States
(12) Patent Application Publication
(10) Pub. No.: US 2010/0228183 A1
(43) Pub. Date: Sep. 9, 2010

(54) METHOD AND APPARATUS FOR THE DEACTIVATION OF BACTERIAL AND FUNGAL TOXINS IN WOUNDS, AND FOR THE DISRUPTION OF WOUND BIOFILMS

(76) Inventor: Gerard V. Sunnen, New York, NY (US)

Correspondence Address: OSTROLENK FABER GERB & SOFFEN, 1180 AVENUE OF THE AMERICAS, NEW YORK, NY, 100368403, US

(21) Appl. No.: 12/753,581

(22) Filed: Apr. 2, 2010

Related U.S. Application Data
(63) Continuation-in-part of application No. 11/110,066, filed on Apr. 20, 2005.

(51) Int. Cl. A61M 35/00; A61L 101/10

(52) U.S. Cl. 604/25

(57) ABSTRACT

An ozone/oxygen treatment system comprising an ozone generator for generating a predetermined ozone/oxygen mixture; and a treatment chamber connected to the ozone generator for receiving and applying the ozone/oxygen mixture to a predetermined portion of a patient's body, the treatment chamber having variable size and shape for enclosing said predetermined body portion and having a structure enabling the treatment chamber to enclose without touching the body portion. Also disclosed is a sensor disposed in the treatment chamber for sensing at least one of ozone concentration, temperature, humidity and bacterial gases. A control unit receives data from the sensor and automatically maintains the ozone concentration and/or heat or humidity at a predetermined range. Arrangements may be provided for directing the ozone to the body portion to be treated, and/or for directing the ozone to the interior and/or underneath a wound biofilm.

### CROSS-REFERENCE TO RELATED APPLICATIONS

The present application is a continuation-in-part of U.S. Ser. No. 11/110,066 filed Apr. 20, 2005, incorporated by reference in its entirety.

### BACKGROUND OF THE INVENTION

1. Field of the Invention

The present invention relates to an apparatus and method for precise ozone/oxygen delivery applied to the treatment of dermatological conditions, including the deactivation of bacterial and fungal toxins in wounds, and for the disruption of wound biofilms, and related disorders.

2. Related Art

Wounds, especially chronic wounds, continue to present daunting obstacles to treatment. Diabetic, decubitus and vascular skin ulcers are manifestations of diseases affecting metabolism and circulation.

Fresh wounds, as seen in accidents, surgical lesions and war trauma, can be remarkably prone to invasion by aggressive bacterial onslaughts. In these scenarios, amputation, with all its attendant bodily and psychological impact, is an all too frequent sequel.

Perennial obstacles of wound resolution are toxins and biofilms. Toxins produced by bacteria and fungi attack host tissues and immune defenses; and biofilms give microorganisms protection from topical therapeutic agents.

### Wound Toxins

A major impediment to wound resolution is infection. Colonizing microorganisms, by sheer population growth, can advance deeper into tissues, moving from epidermis to dermis, and further into connective tissues and bone.

One crucial element in wound resolution involves bacterial and fungal toxins. Toxins are biochemical substances that, as byproducts of microorganism growth, are injurious to host tissues. Toxins can significantly delay healing. Highly poisonous toxins can produce massive tissue breakdown, requiring amputation, and they can cause death. Indeed, bacterial protein toxins are the most potent human poisons known and a major component of bacterial virulence is toxin production.

Endotoxins are biomolecules, usually bacterial membrane lipopolysaccharides, that are released upon bacterial death. Bacterial kill can be the result of antibiotic use, or of host immune defense.

Exposed to endotoxins, host tissues often react with inflammatory responses, which can lead to sepsis.

Exotoxins are substances, usually polypeptides or proteins, actively secreted by bacteria and fungi that destroy host tissues by a variety of mechanisms. They may attack tissue fibrin and collagen via collagenases, hyaluronidases or tryptokinases. They may also act against cell membranes using phospholipases and lecithinases. Exotoxins, also called invasins because they act within the wound to encourage bacterial and fungal growth, are the single most important factor determining morbidity and mortality.

Any chronic wound (e.g., diabetic, decubitus, or vascular skin ulcers, complex surgical, traumatic or war wounds), can harbor numerous families of bacteria and fungi, all capable of emitting toxins. These microorganism families—each with its own profile of susceptibility or resistance to antibiotic agents—secrete different toxins, each with its own mode of noxious action. Common wound invading microorganisms include Staphylococcus, Streptococcus, Clostridium, Pseudomonas, and Corynebacterium, among several others.

### Wound Biofilms

Biofilms are organic layers covering wound surfaces. Produced by many different types of microorganisms, biofilms are complex aggregates of bacteria, fungi and protozoans, in a matrix of proteins, polysaccharides and lipids. Biofilms are noted for the great diversity of organisms that colonize them, and for their complex and dynamic organization.

Far from being static secretions of bacterial byproducts, biofilms are living entities proffering many advantages to colonizing microorganisms, including protection from immune defenses, and from therapeutic agents. Indeed, infections are more prone to fester under biofilms given their shielding capacities.

Disclosed herein is a device, for being gently apposed to wounds, that delivers surface ozone/oxygen—via the ozone generator—not only to the biofilm's outer surface, but also to biofilms' undersurfaces where infections fester. The device has minuscule hollow needles that traverse the biofilm in order to achieve this.

### Ozone as an Anti-Toxin

Toxic bacterial and fungal polypeptides, proteins and lipopolysaccharides, are intrinsically unstable. They can be denatured by a variety of agents such as iodine, sodium hypochlorite, and sodium hydroxide. The majority of these agents, however, are directly toxic to healthy wound tissues, or to the greater host, via systemic absorption.

Topical ozone inactivates all known bacterial and fungal toxins through its remarkable properties as an electron acceptor. Ozone oxidation denatures polypeptides and proteins by forming protein peroxides, and detoxifies lipopolysaccharides by altering lipid molecular configuration.

Ozone has the crucial advantage that in concentrations with which it is apposed to tissues (0.5% to 5% ozone, the rest oxygen), it will not harm them.

### Ozone as a Wound Vasodilator

Vasodilation is important in wound healing because improved circulation brings nutrients and immune factors to host cells. Enhanced circulation also contributes to the removal of microorganisms from the wound site.

Ozone, interfaced with mucous membranes and wounds, reacts with nitrous oxide to form nitric oxide, a potent vasodilator. Indeed, the nitric oxide metabolic pathway is responsible to the vasodilation produced by drugs like sildenafil (Viagra®).

Ozone deactivates bacterial toxins. Ozone also increases wound vascular activity, thus aiding cleaning of the wound site.

### Ozone

Ozone, in its gaseous form, provides superb antipathogenic action for a wide range of bacteria, viruses, protozoa, and parasites. Furthermore, ozone, in appropriately administered concentrations, possesses physiological properties capable of enhancing the healing of tissues.

Ozone, an allotropic form of oxygen, possesses unique properties which are being defined and applied to biological systems as well as to clinical practice. As a molecule containing a large excess of energy, ozone, through yet incompletely understood mechanisms, manifests bactericidal, virucidal, and fungicidal actions which may make it a treatment of choice in certain conditions and an adjunct to treatment in others. The oxygen atom exists in nature in several forms: (1) As a free atomic particle (O), it is highly reactive and unstable. (2) Oxygen (O₂), its most common and stable form, is colorless as a gas and pale blue as a liquid. (3) Ozone (O₃), has a molecular weight of 48, a density one and a half times that of oxygen, and contains a large excess of energy in its molecule (O₃→3/2 O₂+143KJ/mole). It has a bond angle of 127±3, is magnetic, resonates among several forms, is distinctly blue as a gas, and dark blue as a solid. (4) O₄ is a very unstable, rare, nonmagnetic pale blue gas, which readily breaks down into two molecules of oxygen.

Ozone is a powerful oxidant, surpassed in this regard only by fluorine. Exposing ozone to organic molecules containing double or triple bonds yields many complex and as yet incompletely configured transitional compounds (i.e. zwitterions, molozonides, cyclic ozonides), which may be hydrolysed, oxidized, reduced, or thermally decomposed to a variety of substances, chiefly aldehydes, ketones, acids, and alcohols. Ozone also reacts with saturated hydrocarbons, amines, sulthydryl groups, and aromatic compounds.

Importantly relevant to biological systems is ozone's interaction with tissue constituents including blood. The most studied is lipid peroxidation, although interactions have yet to be more fully investigated with complex carbohydrates, proteins, glycoproteins, and sphingolipids.

These properties are responsible for ozone's ability to destroy a wide spectrum of pathogens.

The Effects of Ozone on Pathogens

Infected wounds, and especially chronic lesions, may show a wide spectrum of profuse pathogen growth, including bacteria, viruses, fungi, and protozoa.

The anti-pathogenic effects of ozone have been substantiated for several decades. Its panpathogen properties are universally recognized and serve as the basis for its increasing use in disinfecting municipal water supplies in cities worldwide.

Bacteria

Indicator bacteria in effluents, namely coliforms and pathogens such as Salmonella, show marked sensitivity to ozone inactivation. Other bacterial organisms susceptible to ozone's disinfecting properties include Streptococci, Staphylococci, Shigella, Legionella, Pseudomonas, Yersinia, Campylobacter, Mycobacteria, Klebsiella, and Escherichia coli.

Ozone destroys both aerobic, and importantly, anaerobic bacteria, which are mostly responsible for the devastating sequelae of complicated infections, as exemplified by decubitus ulcers and gangrene.

The mechanisms of ozone bacterial destruction need to be further elucidated. It is known that the cell envelopes of bacteria are made of polysaccharides and proteins, and that in Gram-negative organisms, fatty acid alkyl chains and helical lipoproteins are present. In acid-fast bacteria, such as Mycobacterium tuberculosis, one third to one half of the capsule is formed of complex lipids (esterified mycolic acid, in addition to normal fatty acids), and glycolipids (sulfolipids, lipopolysaccharides, mycosides, trehalose mycolates).

The high lipid content of the cell walls of these ubiquitous bacteria may explain their sensitivity, and eventual demise, in the face of ozone exposure. Ozone may also penetrate the cellular envelope, directly affecting cytoplasmic integrity.

### Viruses

Numerous families of viruses including poliovirus 1 and 2, human rotaviruses, Norwalk virus, Parvoviruses, and Hepatitis B and C, among many others, are susceptible to the virucidal actions of ozone.

Most research efforts on ozone's virucidal effects have centered upon ozone's propensity to splice lipid molecules at sites of viral multiple bond configuration. Indeed, once the lipid envelope of the virus is fragmented, its DNA or RNA core cannot survive.

Non-enveloped viruses (Adenoviridae, Picornaviridae (poliovirus), Coxsachie, Echovirus, Rhinovirus, Hepatitis A, D, and E, and Reoviridae (Rotavirus), have also been studied in relation to ozone inactivation. Viruses that do not have an envelope are called “naked viruses.” They are constituted of a nucleic acid core (made of DNA or RNA) and a nucleic acid coat, or capsid, made of protein. Ozone, in addition to its well-recognized action upon unsaturated lipids, can interact with certain viral proteins and amino acids. Indeed, when ozone comes in contact with capsid proteins, protein hydroxides and protein hydroperoxides are formed.

Viruses have no protection against oxidative stress. Normal mammalian cells, on the other hand, possess complex systems of enzymes (e.g., superoxide dismutase, catalase, peroxidase) which tend to ward off the nefarious effects of free radical species and oxidative challenge. It may thus be possible to treat infected tissues with ozone while respecting the integrity of their healthy cell components.

Herpes viruses are widespread in the human population. Two distinct types of viruses are known, Herpes simplex type I and II, both lipid-enveloped. Type I is transmitted via contact through the mucosa or broken skin (often through saliva), while type II is sexually propagated.

Herpes lesions have been extensively studied with reference to topical ozone administration. Ozone (1) directly inactivates herpes viruses that are lipid-enveloped, (2) acts as a pan-bactericidal agent in cases involving secondary infections, and (3) promotes healing of tissues through circulatory enhancement.

Fungi

Fungi families inhibited and destroyed by exposure to ozone include Candida, Aspergilus, Histoplasma, Actinornycoses, and Cryptococcus. The cell walls of fungi are multilayered and are composed of approximately 80% carbohydrates and 10% of proteins and glycoproteins. The presence of many disulfide bonds has been noted, making this a possible site for oxidative inactivation by ozone.

Protozoa

Protozoan organisms disrupted by ozone include Giardia, Cryptosporidium, and free-living amoebas, namely Acanthamoeba, Hartmonella, and Negleria. The exact mechanism through which ozone exerts anti-protozoal action has yet to be elucidated.

Cutaneous Physiological Effects of Ozone/Oxygen

The positive effects of oxygenation on many dermatological conditions have long been established, and form the basis for the use of hyperbaric oxygen treatment. Oxygen diffuses into the tissues, raising their oxidation-reduction potential, thus inhibiting the growth of anaerobic bacteria.

Ozone greatly supplements the benefits of oxygen administration alone. While the most likely beneficial effect of external ozone administration is pathogen inactivation, it is important to note ozone's contribution to healing through its physiological actions. Ozone dilates the arterioles in wounds, thus stimulating the inflow of nutrients and immunological molecules. By similar mechanisms, the outflow of waste products is accelerated.

Medical Conditions Benefitted By Ozone Therapy

In view of the above-mentioned principles of ozone/oxygen's biological properties, the disclosed methods and apparatus seek to harness this therapeutic potential, not only for the treatment of several dermatological conditions, but also for their prevention.

The following is a list of pathologic sequelae of tissue compromise which may be addressed by external ozone/oxygen therapy. The most serious is gangrene, and the most ominous is gas gangrene.

Gas Gangrene

Gas gangrene may be a rapidly fatal complication of traumatic injuries such as automobile accidents and war injuries, surgical incisions, wounds, burns, and decubitus ulcers, among many other conditions. Predisposing factors include diabetes, arteriosclerosis, lesions associated with colon cancer, surgeries involving the intestinal tract, and septic abortions.

Gas gangrene, also known as necrotizing fascitis, myositis, and myonecrosis is feared because of the rapidity of its evolution and the galloping and irreversible demise of affected tissues.

Several bacterial species are implicated in this process, the most common being Clostridium families. These anaerobic bacteria thrive in the absence of oxygen, feeding on glycogen and sugars, producing lactic acid, and gases such as methane, carbon dioxide, and hydrogen, among others. They also produce toxins causing hemolysis, renal failure, shock, coma, and death, as they are diffused systemically.

Other bacterial species are implicated in gas gangrene aside from Clostridium, including Enterobacteria, E. coli, Proteus, Group A streptococcus, Staphylococcus, Vibrio, Bacteriodes, and Fusiforms. Ozone is effective in inactivating all of these anaerobes and aerobes.

The proposed invention aims at the early detection of the onset of gas gangrene in wounds that are clinically deemed to be potentially at risk, and for early therapeutic responses via calibrated ozone/oxygen infusion.

This is achieved by means of the intra-envelope bacterial gas sensor providing a warning of gas buildup, including, but not limited to, methane, hydrogen, carbon dioxide, indoles, and skatoles, and by the automatic commensurate response through microprocessor-mediated ozone/oxygen infusion into the treatment envelope, at a concentration and for a duration predicated upon programmed treatment protocols.

Infected Wounds

This category of wound has, by definition, not yet reached the status of chronicity due to a combination of circulatory compromise and infective onslaught. In fact, this category of wound may simply be post-surgical, and only potentially prone to infection.

The use of topical ozone therapy in these cases may be solely preventive, aimed at improving circulation on one hand, and inhibiting the proliferation of potentially infective organisms on the other.

Poorly Healing Wounds

Wounds which heal in an indolent manner are frustratingly difficult to master. Generally speaking, poorly healing wounds owe their definition to their chronicity, which is most commonly caused by the profusion and variety of offending organisms they harbor.

War Wounds

War wounds often present complex treatment challenges. Compound fractures are common. Healing is often complicated by the presence of shrapnel and other foreign bodies. Infection is favored by hot weather and high humidity.

Ozone/oxygen external application offer excellent prophylaxis for the infectious processes made likely by the special nature of war wounds.

Decubitus Ulcer

This common condition arises when a patient remains in bed, or in a wheelchair, in a restricted position for a prolonged period of time. The pressure exerted upon skin contact points compresses the dermal arterioles preventing the proper perfusion of tissues. This leads to tissue oxygen starvation, impaired skin resilience, and the eventual breakdown of the skin itself. An expanding ulcer develops, usually infected by a spectrum of pathogenic organisms. At times the breakdown is so severe that the ulcer reaches the bone, ushering in osteomyelitis.

The treatment of decubitus ulcers requires a multidisciplinary approach, including surgical, pharmacological, and physiological interventions. Topical antibiotics often fail to penetrate the depth of the wound, are active only against a limited spectrum of organisms, induce resistance, and not infrequently cause secondary dermatitis in their own right.

Aside from the benefits of topical ozone therapy described in this text, it should be mentioned that an added therapeutic feature of ozone, especially as it relates to the treatment of deep ulcers, is its capability to penetrate into deeper tissue levels, thereby affecting pathogens which would normally be protected by tissue overlay.

Circulatory Disorders

This class of disorders has one common denominator, namely impaired circulation to tissues via compromise of vascular integrity. A prototypic disease is diabetes. Diabetes manifests vascular disturbances to many organ systems (e.g. retina, kidney), and concomitant disruptions to carbohydrate metabolism. In cases where diabetes affects the peripheral circulation, tissues such as the dermis become vascularly compromised, and thus more prone to injuries and infections.

Diabetic ulcers frequently develop following abrasions, contusions, and pressure injuries. These ulcers, not unlike decubitus ulcers, are notoriously difficult to treat. Topical ointments can only address a minor spectrum of putative infectious organisms. These same organisms, furthermore, may rapidly develop antibiotic resistance.

Serially applied ozone topical therapy inactivates most, if not all, offending pathogens and these same pathogens are unable to build a resistance to its effects.

Arteriosclerosis is a condition marked by the thickening and hardening of the vascular tree. The normal pliability and patency of blood vessels is compromised, leading to impaired circulation in many organ systems. In the face of reduced peripheral circulation (e.g., arteriosclerosis obliterans), skin disorders may include trophic changes (e.g., dry hair, shiny skin) apt to injury and eventual ulcer formation.

Lymphatic Diseases

The lymphatic system regulates fluid equilibration within the body and, most importantly, offers infection defense.

Lymphedema is a condition caused by blockage to lymphatic drainage. It may be secondary to trauma, surgical procedures, and infections (e.g., streptococcal cellulitis, filiriasis, lymphogranuloma venereum).

Increasingly common is lymphedema resulting from surgical removal of lymph nodes following surgery for breast cancer. The affected arm in these patients is likely to be chronically swollen and indurated. Exercises are routinely prescribed to develop collateral circulation. Most alarming, however, is the occurrence of infections following even minor injuries to the arm. Injuries are then much more likely to become infected due to the absence of lymphatic system defenses. In these cases, intensive topical wound care is initiated, and systemic antibiotic treatment is prescribed.

Topical ozone treatment applied in a timely fashion to the affected hand or arm may prevent secondary infection; and, it may avoid the need for systemic antibiotics.

Fungal Skin Infections

Fungi are present on human skin in a quasi-symbiotic relationship. Candida, Aspergillus, and Histoplasma, for example, are often found on intact skin, without causing clinical problems.

However, under certain conditions, the normal balance of the dermis is disturbed, allowing superficial fungi to proliferate. Tinea capitis is manifested by pustular eruptions of the scalp, with scaling and bald patches. Tinea cruris is a fungal pruritic dermatitis in the inguinal region.

Serial topical ozone applications have shown marked success in eradicating the most chronic and stubborn fungal skin conditions.

Burns

Thermal burns are divided into first, second, and third degrees, depending upon the depth of tissue damage. First-degree burns are superficial, and include erythema, swelling, and pain. In second degree burns, the epidermis and some portion of the underlying dermis are damaged, leading to blister and ulcer formation. Healing occurs in one to three weeks, usually leading to little or no scar formation.

In third degree burns, muscle tissue and bone may be involved, and secondary infection is common.

It is in cases marked by significant tissue injury, and especially in cases involving infections, that topical ozone therapy finds the most usefulness. In the case of burns, the spectrum of pathogenic organisms may be wide and thus may be ideally suited for ozone therapy.

In burns, externally applied ozone concentrations need to be carefully calibrated. The clinician must be able to gauge the proper ozone concentration geared to the specific medical condition under treatment. In wet burns, for example, initial ozone concentrations will need to be low, in order to prevent inordinate systemic absorption through absorption of exudates. As the burn heals and progressively dries, greater ozone concentrations may then be administered.

Nail Afflictions

Conditions implicating nails which are therapeutically assisted by topical ozone treatment include the following:

1. Candida albicans. Nails in this condition are painful, with swelling of the nail fold, and often, thickening and transverse grooving of the nail architecture. Loss of the nail itself may occur. Another frequent condition is Tinea Unguium, marked by thickened, hypertrophic, and dystrophic toenails. There are currently no topical antifungal agents of proven efficacy for this condition. Systemic anti-fungal agents show a spectrum of noxious side effects.

2. Tinea Pedis (Athlete's Foot). This very common disorder is caused by infection with species of Trichophyton, and with Epidermophyton floccosum. Chronic infection involving the webbing of the toes may evolve to secondary bacterial involvement. Lymphangitis and lymphadenitis may present themselves, as well as infection of the nails themselves (Tinea Unguium; Onychomycosis). Nails may become thickened, yellow, and brittle. The patient may then develop allergic hypersensitivity to these organisms.

Topical ozone therapy offers unique treatment opportunities to these recalcitrant infections. Ozone penetrates the affected areas, including the nails proper, and with repeated administration, is capable of inactivating all species of fungi mentioned above. Healing occurs slowly yet consistently, and skin integrity along with nail anatomy, gradually regain their normal configuration.

Radiodermatitis

This condition occurs during times when the body is exposed to ionizing radiation. This may result from radiological accidents or from radiation therapy. Radiation energy, imparted to cells, leads to cellular DNA injury.

Clinical findings are proportional to the type, amount, and duration of radiation exposure. Several clinical syndromes have been delineated, including Radiation Erythema and Radiodermatitis.

While DNA damage cannot be easily repaired, secondary infections made more likely by decreased tissue resistance may be countered by topical ozone therapy. This avoids the systemic absorption of topical ointments and provides pan-pathogen protection.

Frostbite

Factors contributing to skin injuries due to cold derive from vasoconstriction and the formation of ice crystals within tissues. As frostbite progresses, loss of sensation occurs, and tissues become increasingly indurated to touch. Depending upon length of exposure, dry gangrene may develop. Dry gangrene may then evolve to wet gangrene if infection occurs.

Topical oxygen/ozone therapy has proven to be effective in decelerating or halting the pathogenesis of frostbite through (1) immediate oxygenation of tissues, (2) increasing blood flow through a direct vasodilatory effect upon the dermal arterioles, and (3) prevention of secondary infection.

The method and apparatus provide a microprocessor-controlled intra-envelope milieu geared to the therapy of frostbite, including proper temperature, humidity, and appropriate ozone/oxygen concentrations.

Advantages of Topical Ozone Therapy

Topical ozone/oxygen therapy for the disorders mentioned above requires diagnosis of the underlying conditions, and a correspondingly appropriately tailored treatment plan, which may include any one of several therapeutic modalities utilized concomitantly, including ozone, or may call for the utilization of ozone as the sole therapeutic intervention.

The salient advantages of topical ozone/oxygen therapy include:

1. The ease of administration of this therapy.

2. Ozone is an effective antagonist to the viability of an enormous range of pathogenic organisms. In this regard, ozone cannot be equaled. It is effective in inactivating anaerobic and aerobic bacterial organisms and a wide swath of viral families—lipid as well as non-lipid enveloped—and fungal and protozoan pathogens. To replicate this therapeutic action, the medical conditions in question would have to be treated with complex conglomerations of antibiotic agents.

3. Ozone/oxygen therapy, appropriately applied in a timely fashion, may obviate the need for systemic anti-pathogen therapy, thus saving the patient from the side effects this option could entail.

4. Ozone exerts its anti-pan-pathogenic actions through entirely different mechanisms than conventional antibiotic agents. The latter must be constantly upgraded to surmount pathogen resistance and mutational defenses. Ozone, on the other hand, presents direct oxidative challenge which cannot be circumvented by known mechanisms of pathogen resistance.

Therapeutic ozone/oxygen mixtures applied to external wounds or other dermatological conditions have, to this day, been administered in an imprecise fashion at best. The essential requirement of precise dosing to the rigorous demands of scientific research and to clinical practice has consequently been hampered by this shortcoming.

Externally administered ozone/oxygen mixtures have been applied to the treatment of dermatological conditions since before World War One. The German armed forces fashioned rubber envelopes to surround and seal injured limbs and circulated ozone/oxygen mixtures within them. These mixtures were delivered by field generators because ozone reverts relatively rapidly to oxygen at room temperature, and cannot be stored except at very low temperatures.

Unfortunately, these rubber envelopes frittered easily due to ozone's high oxidative power. Modern materials are available, such as plastics and silicones, that are impervious to oxidation.

A previous treatment system (Sunnen, U.S. Pat. No. 6,073,627), incorporated by reference in its entirety, including its background information, included a transparent envelope with inserted sensors for ozone concentration, humidity, and patient temperature located within the treatment envelope, each relaying data to a display on the ozone generator panel.

U.S. Pat. No. 6,073,627 described an ozone generator which delivered an ozone/oxygen mixture into a treatment envelope encasing the patient's lesion. The problem of delivering a precise ozone/oxygen mixture, however, was only partially solved by this art, based upon the following considerations:

1. The ozone concentration within the treatment envelope was relayed to a readout gauge on the ozone generator, to be read by the clinical personnel. In order to maintain a constant ozone concentration over time and thus adhere to a precise treatment protocol, the personnel would be obliged not only to be present during the entire treatment process but also to adjust the generator's output in response to the fluctuations normally observed in intra-envelope ozone concentrations.

It would therefore be desirable to have a delivery system with an automatic microprocessor-mediated feedback of intra-envelope ozone concentrations in order to counteract their fluctuations in a timely fashion.

2. The temperature of the patient was monitored, but the temperature inside the treatment envelope was not. Intra-envelope ambient temperature is an integral part of the treatment protocol of external wounds with ozone/oxygen. Indeed, some dermatological lesions, such as frostbite, require higher therapeutic ambient temperatures while others do not. Furthermore, temperature itself has an influence upon ozone concentration, with lower temperatures associated with higher concentrations.

It would therefore be desirable to provide a delivery system with a constant integration of ozone and temperature and an automatic microprocessor-mediated feedback of intra-envelope temperature to achieve temperature-to-ozone constancy.

3. Intra-envelope humidity influences ozone concentration, with higher ozone output by the generator needed at higher humidity levels to maintain a constant ozone concentration. The therapy of dermatological conditions requires attention to the maintenance of intra-envelope humidity levels. Some lesions, such as wet gangrene, must be kept dry, while others need moisture. This method and apparatus may comprise an automatic microprocessor-mediated regulation of humidity levels to achieve constancy of the intra-envelope humidity milieu.

4. The space within the treatment envelope can show significant regional variations and fluctuations in ozone concentration, temperature, and humidity, depending upon the placement of probes and the unavoidable presence of pockets of “dead space.” This invention may comprise an intra-envelope fan to homogenize the ambient ozone/oxygen mixture so that probe readings will be accurate.

5. Treatment envelopes in U.S. Pat. No. 6,073,627 were mere plastic bags. They required careful adjustment to anatomical parts so as to minimize unnecessary dead space, offer patient convenience, and avoid apposition of the envelope sheath to the patient's tissues. Described herein are improved envelopes having rigid or flexible supporting ribs or other supporting structures which address these needs.

### SUMMARY

The disclosed method and apparatus address these obstacles. Ozone/oxygen mixtures, properly interfaced with wounds, deactivate bacterial and fungal toxins, and disrupt biofilms.

Also disclosed is a wound care apparatus that presents as a self-stick malleable bubble chamber. The bubble can be configured to adopt a size and shape surrounding the wound surface and its outline.

The treatment bubble preferably contains sensors relaying information on the status of the wound. This includes information on gases produced by colonizing bacteria and fungi that are harbingers of serious clinical sequelae, including gangrene. These gases include, but are not limited to, hydrogen, methane, and carbon dioxide. Other sensors, which may be directly apposed to wound surfaces, detect the presence of bacterial and fungal toxins.

Ongoing information about wound status may be relayed to a microprocessor programmed to respond according to selected treatment protocols.

Responses may include changes in the microenvironment within the treatment bubble, including adjustments in relative ozone/oxygen concentrations, temperature and humidity. Responses may also include the introduction of aerosolized antibiotics or other antimicrobials.

The treatment bubble may also contain a device specifically designed to treat biofilms. This device, gently apposed to the biofilm surface, is provided with needles capable of delivering ozone/oxygen mixtures not only directly to the biofilm surface, but also underneath its surface, thus bypassing the biofilm's protective carapace.

The method and apparatus provide for precise ozone/oxygen delivery applied to the treatment of dermatological conditions, including gas gangrene, and related disorders.

While drugs administered in solid or liquid form are easily quantifiable, drugs in gaseous form present special dosing difficulties, namely the accurate measurement of gas concentration as a function of time of exposure, temperature, and humidity content. Others may need more modulated treatments. In other scenarios, some lesions, in their acute states, may initially require certain dosage administrations, while later in the course of the same treatment session, the required dosage may change.

This disclosure addresses the vital importance of the effective dosing of ozone/oxygen mixtures to the therapy of acutely and chronically infected dermatological lesions. Indeed, without correct dosing of any therapeutic agent, proper medicine cannot be practiced.

The therapeutic action of gaseous ozone/oxygen mixtures derives from the antipathogenic effects, and the beneficial physiological effects, of both ozone and oxygen. However, to be optimally effective, ozone/oxygen mixtures applied to the spectrum of dermatological pathologies must be carefully calibrated.

Disclosed is an ozone delivery system specifically aimed at the treatment of skin pathologies. As such, it is a dermatological ozone/oxygen delivery system.

The wound under treatment is preferably enclosed in an envelope or a self-stick bubble-shaped chamber. The ozone generator delivers a gaseous mixture of ozone and oxygen of various concentrations, predicated on treatment protocols. Ozone concentrations range from 0.1% to 5% by volume.

Humidity content is adjusted to the clinical situation.

The disclosed apparatus further includes a toxin sensor gently apposed to the wound surface. The sensor detects the presence of toxins (e.g., polypeptides, proteins, lipoproteins, lipopolysaccharides). Data from the sensor is relayed to the unit microprocessor which, in turn, gauges appropriate therapeutic responses, predicated on the variety and concentration of toxins detected, and possibly until such time that toxins are no longer detected.

Generator responses comprise changes in proportional ozone to oxygen ratios, humidity content, and length of treatment for each individual session. Computerized monitoring of each serial treatment work toward achieving optimal therapeutic goals.

The apparatus may comprise a further addition, namely a sensor inserted in the treatment envelope capable of detecting gases emitted by pathogenic bacteria growing in the wounds under treatment. These gases are typically observed in gangrenous conditions, including gas gangrene. It is of paramount importance to possess early warning of the development of gangrene, because this condition may evolve so rapidly that the patient's life can be saved only by early amputation. In addition to the early detection of gangrene, this apparatus addresses the early preventive treatment of this potentially fatal sequel of surgical wounds, war wounds, decubitus ulcers, burns, and traumatic injuries.

The apparatus therefore may comprise a microbial gas sensor to monitor the bacterial activity in the wound under treatment. The presence and concentration of pathogen-generated gases are relayed to the generator which, via microprocessor-mediated feedback, modifies the envelope milieu and the duration of the treatment.

Microprocessor-mediated feedback allows the ozone concentration, the humidity, and the temperature within the treatment envelope to be automatically maintained at predetermined and constant levels, if so chosen, or alternatively, to respond to the changing parameters of the wounds under treatment. The sensors within the envelope may thus provide feedback data to modify:

1. The generator's output of ozone concentration via the automatic regulation of oxygen flow through the system and/or the regulation of electrical or other energy applied to the medical grade oxygen for conversion to ozone.

2. The generator's humidity control to satisfy the treatment's humidity requirement.

3. The generator's heat control output.

The automatic feedback-mediated adjustment of these parameters avoids the need for the clinician's constant monitoring of the treatment process. Since treatment duration times range anywhere from a few minutes to several hours or more, it is cumbersome to oversee and hand-regulate delivery system functions in response to the readings of envelope sensors. Such adjustments are not only cumbersome; they make for significant dosage inaccuracies over the range of the treatment session.

The treatment session may be further automated by means of a timer, in software or freestanding, which may (1) shut off ozone delivery to the envelope once the predetermined treatment time has elapsed; (2) shut off ozone delivery to the envelope once the bacterial gas sensors have signaled to do so; or (3) withdraw ozone/oxygen from the envelope while simultaneously infusing it with oxygen, thus signaling the termination of the treatment process.

The treating personnel may then remove the envelope at some time after the treatment cycle is completed. The advantage of this automated process lies in the fact that precise termination of treatment is not predicated upon the constant presence of treatment staff.

Other features and advantages of the method and apparatus will become apparent from the following detailed description of embodiments which refers to the accompanying drawings.

### BRIEF DESCRIPTION OF THE DRAWINGS

FIG. 1 shows a lateral, partially schematic view of a treatment bubble and a wound;

FIG. 2 is a plan view of the apparatus of FIG. 1;

FIG. 3 is a schematic drawing of a toxin deactivation unit and a wound;

FIG. 4 shows schematically a biofilm destructor disposed on a wound having a biofilm;

FIG. 5 shows schematically the configuration of apparatus according to another embodiment, and its use in a system for external O₃/O₂ treatment of an infected leg;

FIG. 6 shows the infected leg and the treatment envelope in more detail; and

FIG. 7 shows another example of a treatment envelope, for the patient's midsection.

### DETAILED DESCRIPTION OF EMBODIMENTS

First Embodiment

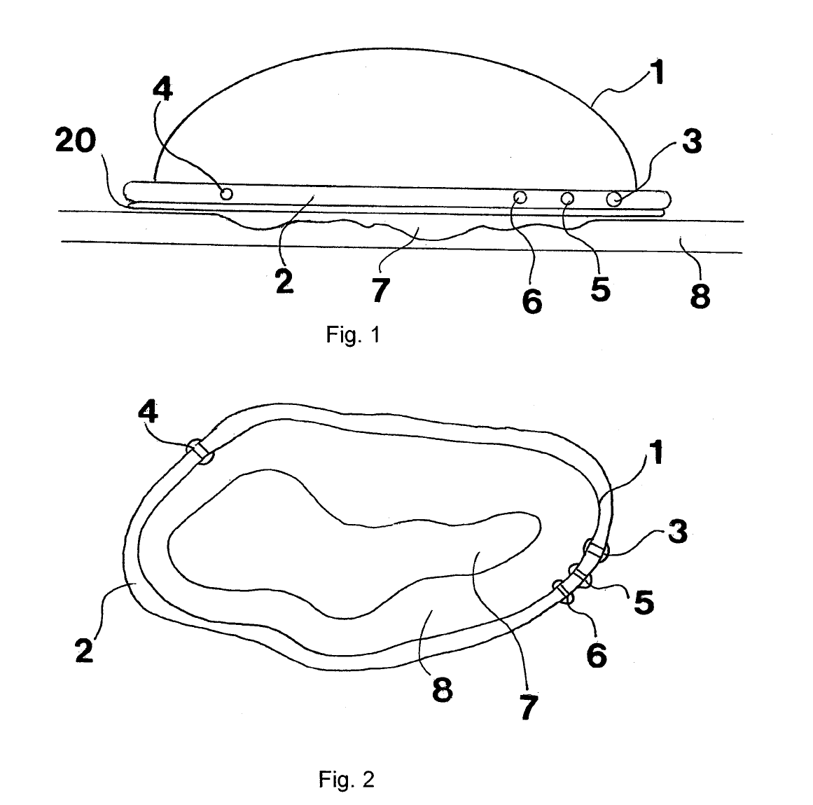

FIG. 1 shows a lateral, partially schematic view of a treatment chamber (1) according to an embodiment, having a malleable rim (2) which is capable of conforming to the outside shape of the wound (7). The inferior rim of the bubble is provided with an adhesive (20), for securing a hermetic seal with the skin (8) surrounding the wound (7). Ozone/oxygen from an ozone generator (not shown) enters through an entry port (3). Gas exits via an exit port (4) to enter an ozone destructor (not shown). Also shown are a toxin sensor gas port (5) and a biofilm destructor gas port (6).

FIG. 2 is a top view of the apparatus of FIG. 1. It shows the treatment bubble (1) conforming to the wound (7) outline.

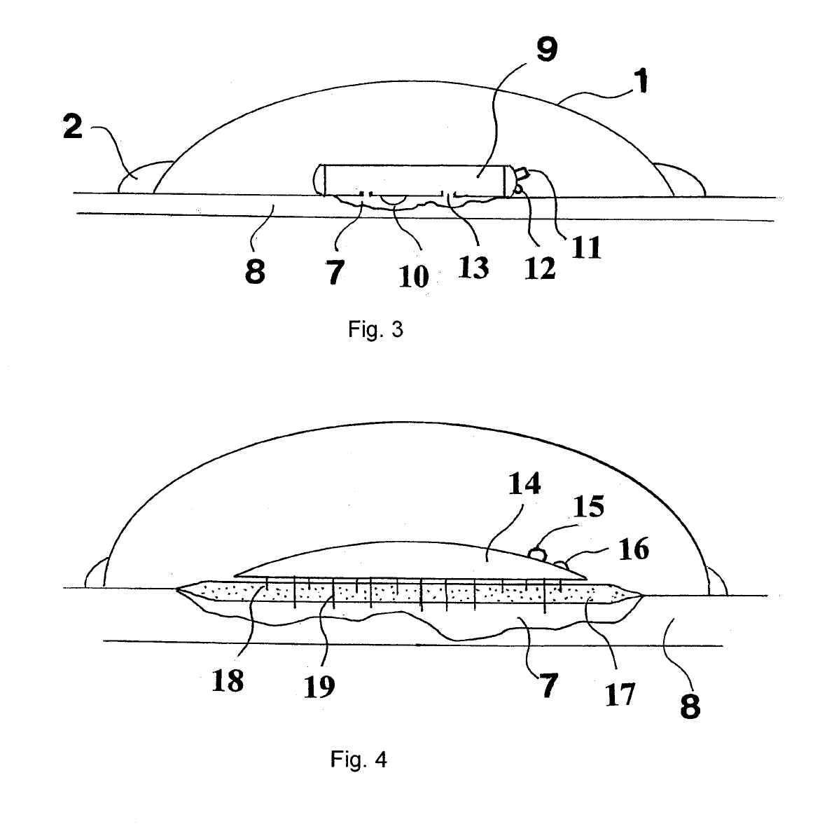

FIG. 3 shows a toxin deactivation unit (9), apposed to the wound (7) surface. Ozone/oxygen enters via the entry port (11). Ozone is provided to the wound via ozone outlets (13). An ozone sensor (10) relays ozone concentration to a microprocessor (not shown). Also shown is an ozone/oxygen sensor port (12).

FIG. 4 shows a biofilm destructor (14) which receives ozone/oxygen via an entry port (15) and delivers it to the wound biofilm (17) through needles (18, 19). In this example, the needles (18) are relatively short and the needles (19) are relatively long, so as to deliver the ozone to both the interior of the biofilm (17) and to the wound (7) region below the biofilm (17). Also shown is an O₃ concentration detector port (16).

Second Embodiment

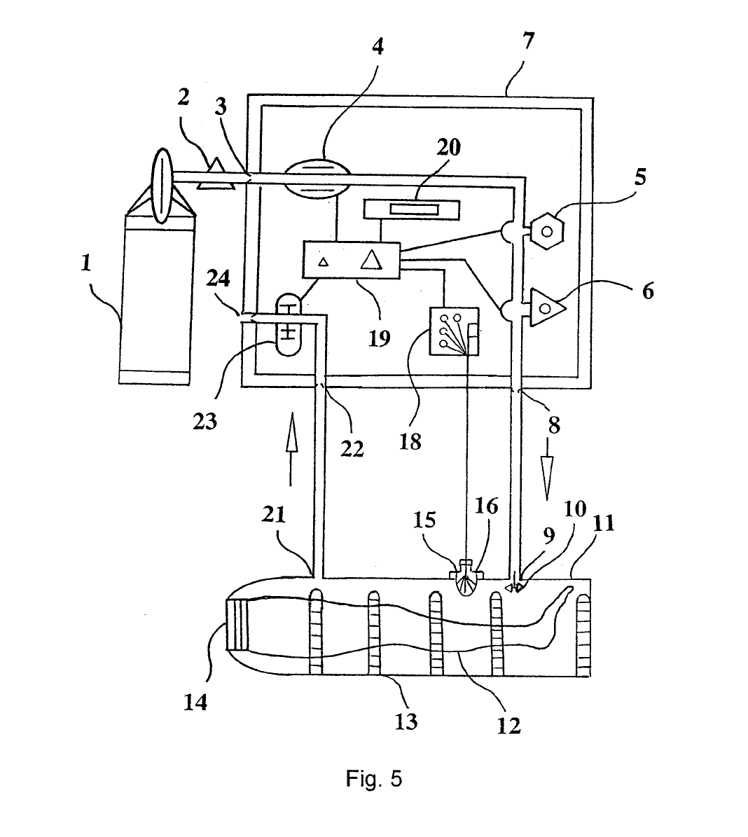

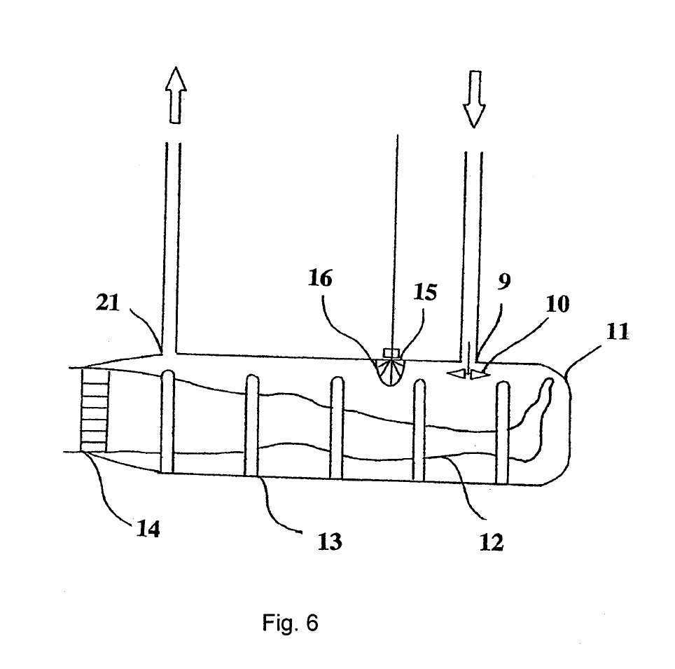

FIG. 5 shows schematically the configuration of apparatus according to another embodiment, and its use for the external O₃/O₂ treatment of an infected leg.

For additional description of this embodiment, including technical and medical background material, see Ser. No. 11/110,066 filed Apr. 20, 2005, incorporated by reference in its entirety.

The medical grade oxygen tank (1) feeds oxygen through a regulator (2) and enters the ozone generator (7) through an intake valve (3).

A power unit (4) imparts electrical energy for converting the oxygen to ozone.

The O₂/O₃ mixture passes through a humidifier (5), then through a heater/cooler (6), exiting from the generator outflow valve (8) to enter the inlet (9) of the treatment envelope (11). An intake fan distributor (10) serves to homogenize the intra-envelope gas milieu.

The treatment envelope (11) encases the affected limb (12). Supporting ribs (13) hold the treatment envelope in a manner to prevent the sheath of the envelope from contacting the skin of the patient.

The envelope forms a hermetic seal (14) with the limb. This may be accomplished by means of a Velcro (R) or adhesive seal.

The envelope contains an opening (15) through which is inserted a multi-sensor head (16) containing sensors for ozone concentration, oxygen concentration, temperature, humidity, and the presence of bacterial gases.

These sensors relay their signals to their respective analyzers, which are grouped in the analyzer unit (18).

All the above analyzers project their data to the microprocessor (19).

The microprocessor connects with the LCD (liquid crystal display) (20), to provide a digital readout of the data at hand.

The microprocessor, in addition, has reciprocal relationships with the power unit (4), the humidifier (5), the heater/cooler (6), and the analyzer unit (18).

Ozone/oxygen exits the treatment envelope through the envelope outlet valve (21) and enters the ozone generator (7) through its envelope effluent intake valve (22), and on to the ozone destructor (23) which de-energizes the remaining ozone, converting it to oxygen. This oxygen may safely exit the ozone generator through its exit valve (24).

As seen in FIG. 6 the treatment envelope (11) encases the affected limb (12). The envelope hermetically seals the limb at (14) using a Velcro (R) or adhesive fastener, for example.

Ribs (13) within the envelope keep it from collapsing. They prevent the envelope membrane(11) from touching the skin of the patient. The ribs shown are circumferential of the generally cylindrical envelope, but could take any other suitable configuration.

The envelope is provided with an entry port (15) for the easy insertion and removal of the multi-sensor head (16) from the ozone generator.

The multi-sensor head contains sensors including an ozone sensor, an oxygen sensor, a temperature sensor, a humidity sensor, and a bacterial gas sensor.

The ozone/oxygen mixture enters the envelope through inflow valve (9). A fan (10), incorporated in or near the inflow valve, works to homogenize the intra-envelope milieu. Gas exits the treatment envelope through its exit valve (21) for processing by the generator.

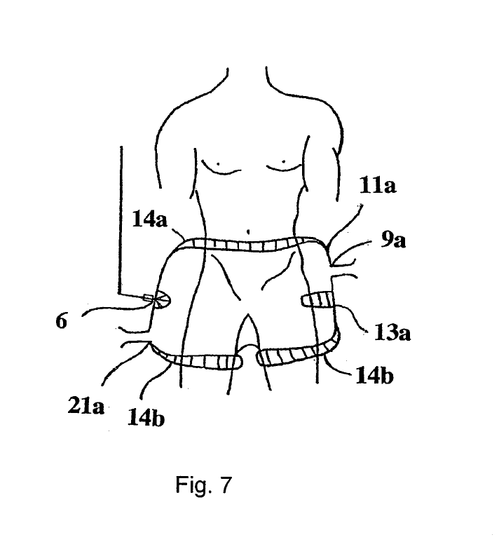

In FIG. 7, the treatment envelope (11a) shows a specialized configuration in the form of briefs. It is fitted with supporting ribs (13a), which keep the membrane of the briefs away from the patient's skin. The envelope hermetically seals the torso and legs by means of adhesive or Velcro® fasteners (14a, 14b).

Ozone/oxygen enters the envelope via its entry port (9a). The gas exits through the envelope exit port (21a), to join the ozone generator where it will be converted to oxygen.

The multi-sensor head (16) relays data about the intra-envelope ozone milieu to the analyzers and to the microprocessor in the generator.

The foregoing has described a method and apparatus for the deactivation of wound bacterial and fungal toxins, including but not limited to endotoxins and exotoxins via the use of ozone/oxygen mixtures. Ozone/oxygen mixtures neutralize toxins via their great oxidizing properties. Bacterial and fungal toxic polypeptides, proteins and lipopolysaccharides, are intrinsically unstable. Ozone oxidation denatures polypeptides and proteins by forming protein peroxides; and lipopolysaccharides by altering their lipid molecular configurations.

The method and apparatus are effective for the resolution of wounds, acute and chronic (diabetic, decubitus and vascular ulcers; surgical wounds, traumatic and war wounds), using ozone's capacity to improve wound circulation via the activation of the nitric oxide pathway.

A method of toxin detection is also described, utilizing a sensor probe directly or indirectly apposed to the wound surface. This sensor has the capacity to detect polypeptide, protein and lipopolysaccharide toxic molecules, among others. The toxin sensor determines toxin presence and concentration on the wound under treatment. Data from the sensor is relayed to a microprocessing unit. Programmed to respond to the detection of toxins, the unit commands the ozone generator to emit an ozone/oxygen gaseous mixture whose relative ozone to oxygen concentration is adjusted for the situation at hand. The unit, for example, could be programmed to continue the treatment until toxins are no longer detected, or for a predetermined time. Gradients of toxin presence trigger commensurate ozone/oxygen responses of preferably at least 0.1% by volume, and more usually at least 0.5% by volume. At maximal toxin presence, ozone concentrations may reach 5% by volume.

A toxin deactivation unit is provided, which is directly apposed to the wound. This unit may incorporate the toxin sensor. This unit receives ozone/oxygen mixtures from the ozone generator, and via opening on its undersurface, delivers them directly to the wound.

A self-adhesive treatment chamber is configured for encasing a wound, adapting itself to the configuration of the wound. As such, it is malleable, its inferior edge susceptible of adopting chosen shapes commensurate with wound morphology. Its inferior edge has a biomedical adhesive that provides it with an airtight seal to the skin. A transparent dome-like covering tops the chamber. The apparatus may be made of ozone-resistant material such as silicone, and has ports to allow entry of ozone/oxygen gaseous mixtures and, if so chosen, aerosolized therapeutic agents such as antibiotics. The same or analogous port may be used to connect the biofilm removal device to the ozone generator. The chamber also has ports for connecting toxin sensors from the wound surface to the microprocessor unit. The chamber has an opening for removal of gases within it, channeled to the ozone destructor, for the conversion of ozone to oxygen.

A biofilm removal device is also provided, for being apposed directly on the wound under treatment and within the bubble chamber. Its hypoallergenic ozone resistant surface is punctuated with minuscule hollow needles (for example 23 to 36 gauge hollow needles). The needles are of variable length. Some needles are very short to allow penetration only within the substance of the film. Other needles are longer and reach the undersurface of the biofilm. Ozone enters the device via tubing from the ozone generator. Once in the device, ozone courses through the needles to attack biofilm constituents, both within the film itself, and under its surfaces. Ozone neutralizes microorganisms, deactivates biofilm toxins, and oxidizes organic molecules within the biofilm. With a single, or repeated use, the biofilm is destroyed, paving the way for accelerated wound healing.

Although particular embodiments have been described, many other variations and modifications and other uses will become apparent to those skilled in the art. Therefore, the present invention is not limited by the specific disclosure herein.

What is claimed is:

1. An ozone/oxygen treatment system comprising:
an ozone generator for generating a predetermined ozone/oxygen mixture; and
a treatment chamber connected to said ozone generator for receiving and applying said ozone/oxygen mixture to a predetermined portion of a patient's body,
said treatment chamber having variable size and shape for enclosing said predetermined body portion and having a structure enabling said treatment chamber to enclose without touching said body portion.

2. The system of claim 1, further comprising a device for forming an air tight seal between said treatment chamber and said body portion.

3. The system of claim 1, further comprising a bacterial toxin sensor disposed within said treatment chamber.

4. The system of claim 3, further comprising a control system receiving data from said toxin sensor and controlling said ozone/oxygen mixture in response thereto.

5. The system of claim 1, further comprising a fan disposed within said treatment chamber.

6. The system of claim 1, further comprising a sensor disposed in said treatment chamber for sensing at least one of ozone concentration, temperature, humidity and bacterial gases.

7. The system of claim 6, further comprising a control unit receiving data from said sensor and in response to said data, automatically controlling said ozone generator to maintain said ozone concentration at a predetermined range.

8. The system of claim 7, wherein said ozone concentration is substantially 0.1-5% by volume.

9. The system of claim 8, wherein said ozone concentration is at least 0.5% by volume.

10. The system of claim 7, further comprising apparatus for supplying at least one of heat and humidity to said treatment envelope,
said apparatus being automatically controlled by said control unit to maintain said heat and/or humidity at a predetermined range.

11. The system of claim 7, wherein said control unit is operative for controlling said ozone concentration as a function of time.

12. The system of claim 1, further comprising a toxin deactivation unit for being apposed to said body portion within said chamber.

13. The system of claim 12, wherein said toxin deactivation unit receives said ozone/oxygen mixture and channels it directly to said body portion via outlets in a surface apposed to said body portion.

14. The system of claim 12, further comprising a toxin sensor in said toxin deactivation unit.

15. The system of claim 1, further comprising a biofilm removal device for being apposed to a biofilm at said body portion within said treatment chamber, for delivering said ozone/oxygen mixture at least to the interior of said biofilm.

16. The system of claim 15, wherein said biofilm removal device further delivers said mixture to said body portion beneath said biofilm.

17. The system of claim 16, wherein said biofilm removal device has a plurality of needles of respective lengths for delivering said mixture to said biofilm interior and to said body portion beneath said biofilm.

18. A method of treating a predetermined body part with an ozone/oxygen mixture, comprising the steps of:
generating a predetermined ozone/oxygen mixture; and
supplying said ozone/oxygen mixture to a treatment chamber enclosing said predetermined body part,
providing said treatment chamber with variable size and shape for enclosing said predetermined body part and having a structure enabling said treatment chamber to enclose without touching said body part.

19. The method of claim 18, further comprising a device for forming an air tight seal between said treatment chamber and said body portion.

20. The method of claim 18, further comprising the step of sensing bacterial toxins within the chamber.

21. The method of claim 20, further comprising the step of controlling said ozone/oxygen mixture supply in response to said toxins sensed within said chamber.

22. The method of claim 18, further comprising the step of circulating said ozone/oxygen mixture within said treatment chamber.

23. The method of claim 18, further comprising the step of:
sensing at least one of ozone concentration, temperature, humidity and bacterial gases within said treatment envelope.

24. The method of claim 23, further comprising the step of receiving data from said sensor, and in response to said data, automatically controlling said ozone generator to maintain said ozone concentration at a predetermined range.

25. The method of claim 24, wherein said ozone concentration is substantially 0.1-5% by volume.

26. The method of claim 25, wherein said ozone concentration is at least 0.5% by volume.

27. The method of claim 24, further comprising the steps of supplying at least one of heat and humidity to said treatment chamber, and maintaining said heat and/or humidity at a predetermined range.

28. The method of claim 24, further comprising the step of controlling said ozone concentration as a function of time.

29. The method of claim 23, further comprising the step of providing an antibiotic through an opening in said treatment chamber.

30. The method of claim 18, further comprising the step of channeling said ozone/oxygen mixture directly to said body portion via a plurality of outlets in a toxin deactivation unit apposed to said body portion.

31. The method of claim 30, further comprising the step of sensing bacterial toxins at said toxin deactivation unit.

32. The method of claim 18, further comprising the step of providing said mixture at least to the interior of a biofilm.

33. The method of claim 32, further comprising the step of providing said mixture to said body portion beneath said biofilm.

34. The method of claim 32, wherein said biofilm removal device has a plurality of needles of respective lengths for delivering said mixture to said biofilm interior and to said body portion beneath said biofilm.
